(19) United States
(12) Patent Application Publication
(10) Pub. No.: US 2005/0232959 A1
(43) Pub. Date: Oct. 20, 2005

(54) Item with decorative voids

(76) Inventor: Jeffrey Bell, Alpha, NJ (US)

Correspondence Address: REED SMITH LLP, P.O. BOX 488, PITTSBURGH, PA, 15230-0488, US

(21) Appl. No.: 11/141,974

(22) Filed: May 31, 2005

Related U.S. Application Data
(63) Continuation of application No. 09/971,739, filed on Oct. 4, 2001, now abandoned.
(60) Provisional application No. 60/237,882, filed on Oct. 4, 2000.

(51) Int. Cl. A61L 9/04; A61K 7/00

(52) U.S. Cl. 424/401

(57) ABSTRACT

A gel-based or solid item with one or more decorative voids. Gel-based and solid compositions typically used for air fresheners or candles permit the inclusion of shaped objects that, when removed once the composition cures sufficiently, create decorative voids. Depending upon the shape of the objects used, these voids may possess a high degree of detail. Highly detailed decorative objects often produce an ice sculpture-like appearance to the decorative void. Additional effects, such as glitter or a pearl-like appearance, may be added to enhance the appearance of the decorative void.

### CLAIM OF PRIORITY

This application claims the benefit of the earlier filing date of provisional application No. 60/237,882 filed on Oct. 4, 2000.

### BACKGROUND OF THE INVENTION

1. Field of the Invention

The present invention generally relates to gel-based and solid composition items, and, more specifically, the present invention relates to gel-based and solid composition items such as air fresheners and candles with decorative voids therein, and methods for making the same.

2. Description of the Background

Compositions that release fragrant volatile components into the air have long been used as air fresheners and candles. Traditionally, such compositions have been valued for their utilitarian ability to impart a desired scent into the air and/or to mask stale or unpleasant odors. Within the household, they have found their most common use in areas where stale or unpleasant odors are most likely to occur, such as bathrooms and kitchens. The containers or dispensers used to dispense these compositions into the air have tended to be more functional than attractive.

More recently, both scent-releasing and non-scent releasing compositions have come to be used to extend a room's decor or theme into an additional sensory dimension or to evoke a particular feeling or mood. For example, the scent of aromatic woods may be used to bring the feeling of the outdoors into the home, or a spicy apple scent may be used to induce nostalgic recollections of home-baked pies. The scents used in these compositions typically are more complex and sophisticated than the heavily perfumed, disinfectant-type scents commonly used in traditional air freshening compositions or candles.

The trend toward the use of scented compositions to enhance home decor and ambiance has resulted in an increased use of these scented compositions in areas of the household, such as bedrooms and living rooms, where air fresheners traditionally have been less frequently used. Persons who wish to use scented compositions in these areas of the home typically prefer an aesthetically pleasing item that harmonizes with, or even enhances, household decor. Such aesthetic benefits are often desired even without the use of a scented composition.

In particular, gel or solid compositions have been used for both air fresheners and candles because of the versatility of such compounds. For example, a colored or lightly-colored composition may be used to expose interior decorations or to allow a greater amount of light to pass through or be emitted from the item. Clear compositions in these items may allow a maximal amount of light to be emitted.

Various gel-based and solid compositions may be used to create these items. For example, U.S. Pat. No. 6,071,506 to Semoff et al., incorporated herein by reference, discloses a transparent gel composition used as an air freshener. Likewise, U.S. Pat. No. 5,879,694 to Morrison et al. and U.S. Pat. No. 5,578,089 to Elsamaloty, both of which are incorporated herein by reference, also disclose a transparent gel candle. At least some of these gel candle bodies are clear, but may be colored or scented. These incorporated references teach various compositions that are exemplary of those that may be used for a gel item with a decorative void.

Solid candles and/or air fresheners may also be adapted according to the present invention to incorporate one or more decorative voids therein. For example, U.S. Pat. No. 6,111,055 to Berger et al. discloses several clear solid compositions. These compositions may be used in the manufacture of a clear candle or air freshener. The gel-based and solid candle compositions described above may be used with the methodologies of the present invention to produce items with decorative voids. Similar compositions that result in items with similar characteristics to these examples may also be used and are well known to one skilled in these arts.

Various attempts have been made to make gel items more aesthetically pleasing. U.S. application Ser. No. 09/329,702 discloses a gel candle comprising an inner portion and outer portion, the outer portion containing decorative inserts such as silk leaves, crystals, stones and glitter. U.S. Patent No. Des. 411,891 discloses a gel candle having various shapes and designs embedded therein. U.S. Patent No. Des. 387,446 discloses a candle containing bubbles. U.S. Pat. No. 6,071,506 discloses a transparent gel air freshener in which solids such as botanicals may be embedded or suspended. These botanicals include berries, slices of fruit, leaves, seeds, flowers, herbaceous sprigs, small branchlets and the like.

### SUMMARY OF THE INVENTION

In at least one preferred embodiment, the present invention comprises an approach to enhance the aesthetic appearance of air fresheners, candles, and other items. These items may be made from a gel-based composition or a solid composition. Previously, objects have been included in a gel candle to improve the appearance. These objects ranged from various shapes (as in U.S. Des. Pat. No. 411,891) to botanicals (as in U.S. Pat. No. 6,071,506). The present invention takes the novel approach of incorporating decorative voids.

The present invention also includes a variety of methods that may be used to produce these items with decorative voids. In one presently preferred embodiment, the composition is partially poured into a mold and a decorative object is inserted into the mold to a desired position. For example, the object may be inserted until a lower face of the shape is flush with the top of the poured composition. After at least partial curing of the composition, the decorative object may be removed to reveal a decorative void in the composition. If the composition is fully cured and removed from the mold, the remaining gel-based or solid composition may be inverted to partially encapsulate the decorative void. If the decorative object has a defined top or upper portion, the object may be inserted into the composition upside-down or inverted, so that the final decorative void will be oriented correctly. For a different effect, an additional amount of the same or a different composition may be poured over the top of the partial pour before the object is removed to produce an item with a fully embedded object. The decorative shape may then be removed from the item after at least partial curing of the composition. This removal may occur by cutting a slit in or folding away the second layer of a “rubbery” composition. The removal may also be employed by cutting away a “plug” from the second composition layer, removing the object, and then reinserting the lower layer plug to produce the embedded decorative object. The result preferably includes a gel-based or solid item with one or more decorative voids.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 illustrates a gel item with a decorative void in the shape of a star;

FIG. 2 illustrates a gel candle with a decorative void in the shape of a star;

FIG. 3 illustrates a decorative star shape that may be used to create the void in the gel items of FIGS. 1 and 2;

FIG. 4 illustrates a partially poured gel item with a decorative star shape inserted into the top of the partially poured gel item;

FIG. 5 illustrates a completely poured gel item with a decorative star shape at least partially within the gel item;

FIG. 6 is a front view of an item with a decorative void in the shape of a cat;

FIG. 7 is a side view of an item with a decorative void in the shape of a dog;

FIG. 8 is a side view of an item with a decorative void in the shape of a fish;

FIG. 9 is a top view of an item with a decorative void in the shape of a fish;

FIG. 10 is a side view of an item with a decorative void in the shape of a fish with a lower pan; and

FIG. 11 is a top view of an item with a decorative void in the shape of a rose.

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. Those of ordinary skill in the art will recognize that other elements are desirable and/or required in order to implement the present invention. However, because such elements are well known in the art, and because they do not facilitate a better understanding of the present invention to those skilled in the art, a discussion of such elements is not provided herein. The detailed description will be provided hereinbelow with reference to the attached drawings.

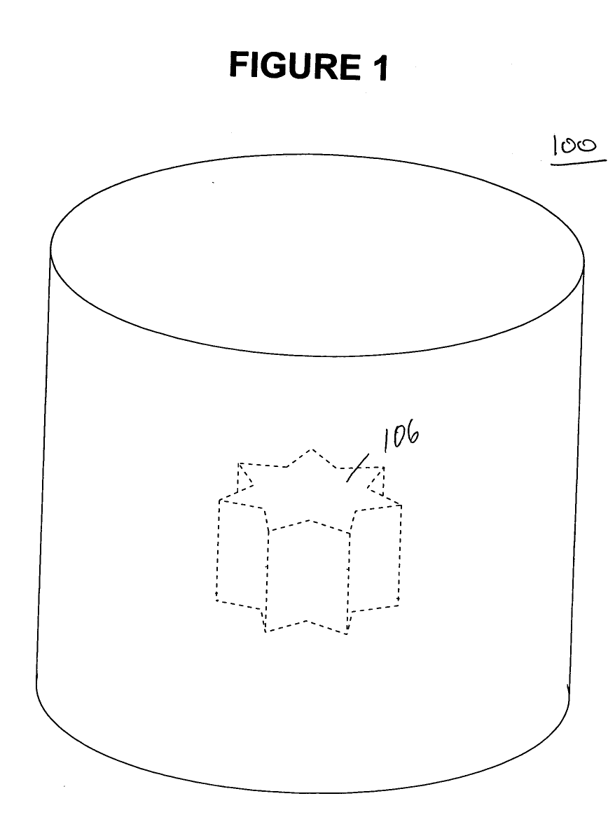

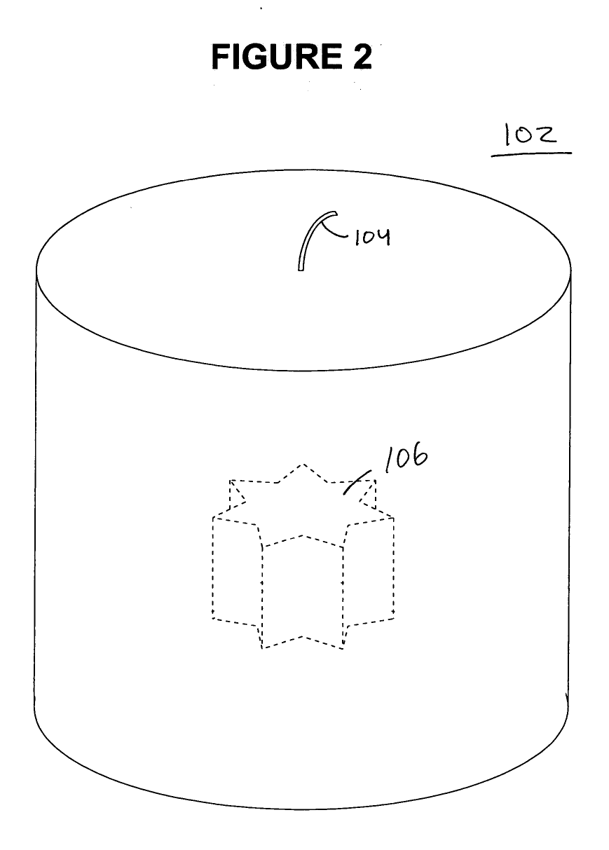

Water-based and oil-based gel compositions, and methods for making such compositions, used for gel air fresheners and gel candles, have been described in the art. See e.g., U.S. Pat. No. 6,071,506; U.S. Pat. No. 5,578,089; U.S. Pat. No. 5,879,694. Solid compositions and methods of making such compositions, have also been described in the art. See, e.g., U.S. Pat. No. 6,111,055. The gel or solid items with decorative voids described and claimed herein can be used with the known gel-based or solid compositions and production processes. An exemplary air freshener 100 and candle 102 with wick 104 are shown in FIGS. 1 and 2 respectively.

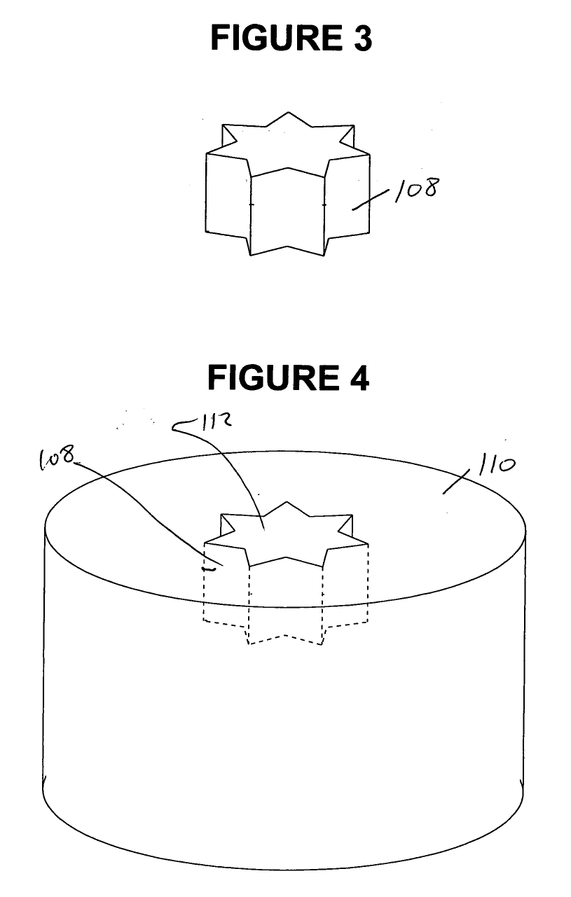

The decorative voids 106 are created in the gel-based or solid items 100, 102 by temporarily inserting objects into the gel or solid before it cures. The decorative objects 108 may be of virtually any shape and are shown as a three-dimensional star (FIG. 3) by way example only. The objects 108 may be made of plastic, resin, porcelain or any suitable material. Objects 108 that are flexible or deflatable also may be used, and may increase the ease with which the object 108 is removed. For gel processes that involve heating the gel composition, the decorative objects 108 should be made of a material that will not be adversely affected by the heat of the process. For example, an ice-based or wax-based decorative object 108 may be used in some embodiments, but would not be preferable for use with certain higher temperature methodologies. The ice or wax-based decorative object 108 may not necessitate the removal of the shape after curing because some or all of the shape may evaporate or otherwise dissipate.

As shown in FIG. 4, the decorative object 108 is preferably at least partially embedded in a gel-based or uncured solid composition 110 that may be supported by a pouring mold (not shown). In FIG. 4, the object 108 is substantially even with the surface of the composition. This is considered the “lower” face 108 because the items with decorative voids are often inverted after manufacture.

After the gel-based or solid composition 110 cures sufficiently, the object or objects 108 may be removed, leaving behind the void made from the impression of the object 108 that previously occupied the void. Where the decorative object 108 has particular surface detail, such as fur on a dog figurine or scales on a fish figurine, the void typically possesses those details in the impression left in the gel or solid composition. The composition is preferably clear (or substantially clear) thereby making a decorative void which is visible from the exterior of the gel-based or solid item. In many instances, the decorative void has an ice sculpture-like appearance.

In some instances, the decorative object 108 may be inserted into the composition 110 inverted from the way in which the finished product will appear. When the decorative object 108 is removed, once the gel or solid has sufficiently cured, the item is placed right-side-up (it is inverted) so that the decorative void also appears in the proper orientation. This is particularly important where the decorative voids have a desired orientation (such as a dog or a cat); it becomes less important where the orientation does not matter (such as stars or spheres). The inverted void will now be embedded in the composition 110 on all but one side.

Additional effects may be added to enhance the appearance of the decorative void. For example, prior to insertion in the gel or solid 110, the decorative object 108 may be coated with glitter (or coloring) so that, once inserted and then later removed, the glitter will remain embedded in the gel or solid boundaries of the decorative void. Alternatively, glitter may be introduced into the void after the object 108 is removed. Materials to create other effects, such as a pearl-like appearance, may be used in a similar way. Moreover, the decorative voids may be used in conjunction with embedded objects to create desired effects.

Where gel has elastic properties, such as in the gel compositions gel described in U.S. Pat. No. 5,871,765 and U.S. Pat. No. 4,369,284, the gel can simply be poured over and around the object 108 (e.g., the object 108 can be fully submerged in the composition). Once the gel has cooled, the gel can be deformed, slit, stretched or pulled away from the object 108, and the object can be removed. Preferably, the gel will return to its molded shape, leaving the impression (void) of the decorative object 108.

With solids or gels with limited elasticity such as those in U.S. Pat. No. 6,111,055 and U.S. Pat. No. 5,679,334, one option is to use a decorative object 108 that has its widest dimension situated at the surface of the gel (the lower face 112 of the object 108). Once the gel or solid has cooled, the object 108 can simply be removed since the wider portion of the object does not have to pass through the body of the gel/solid. Therefore, minimal disturbance to the impression should occur. For decorative objects 108 that have an orientation whereby the bottom is typically wider than the top (such as a pyramid) the gel/solid can be inverted after removal of the object to allow the proper orientation.

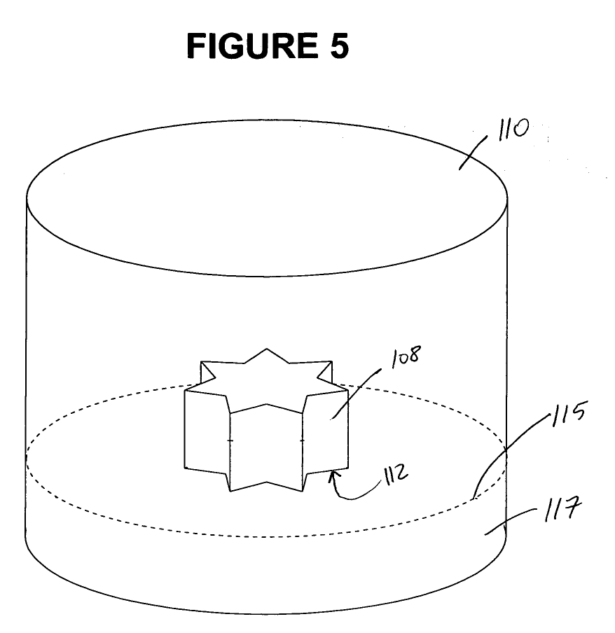

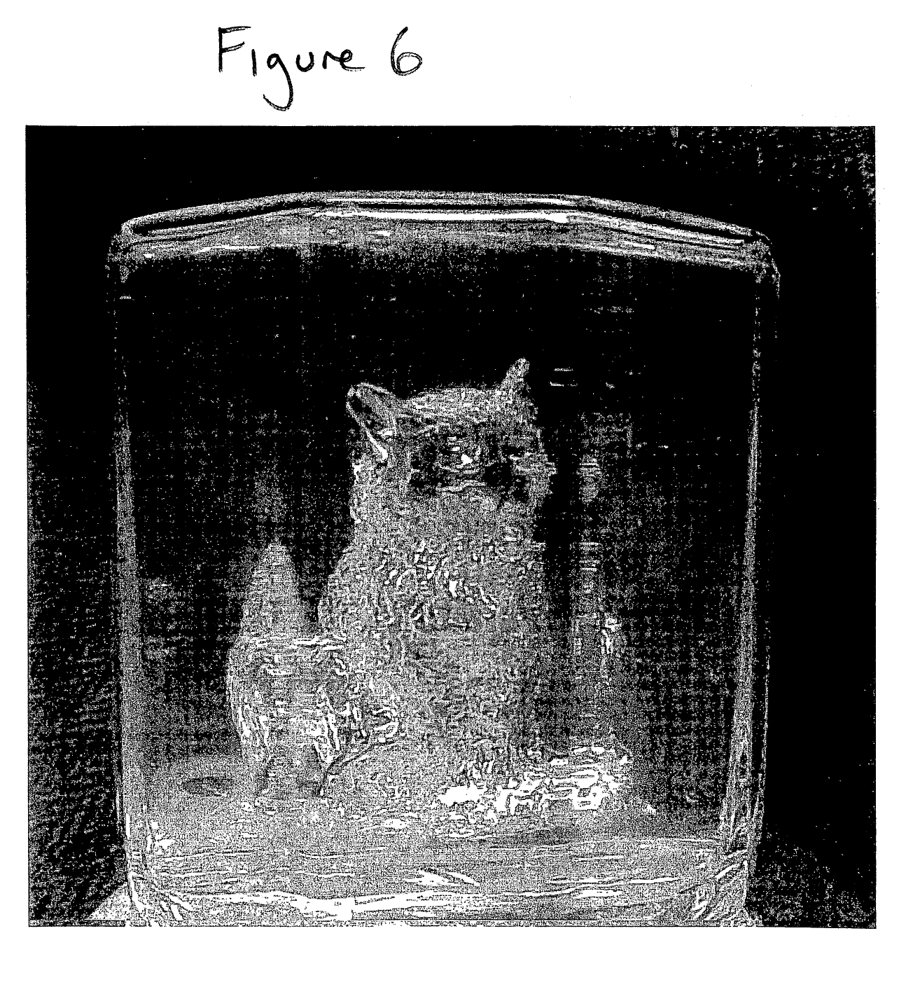

For the gels/solids compositions 110 with limited elasticity as described above, as there may be a desire to have the object 108 completely embedded in the composition 110 or it may be impossible to have the widest part of the object at the surface 112. In these cases, an opening or plug can be cut in the composition allowing the object 108 to be removed. Specifically, FIG. 5 shows an example of shearing 115 a lower portion 117 from the composition 110, thereby exposing the surface 112 of the object 108. The object 108 can then be removed. Once the object is removed, the lower portion 117 or the smaller “plug” not shown can be reinserted. The composition can be left as it is, or can be placed in a larger container, and an additional composition layer may be poured over the top.

The completed item with decorative void may be placed in a container (typically clear) such as a glass or a jelly jar, or it may stand alone without a container. Where the item is a candle, the decorative voids are preferably placed such that they do not interfere with the safe burning of the candle.

In certain instances, it may be preferred to over-pour the item with additional gel to improve the appearance of the gel item, or to fill any voids in the gel item or between the gel item and any container in which the final product resides.

The decorative void itself could also be separately decorated after it is made. For example, once the decorative object is removed from the composition and a void remains, the void could be filled with a liquid or other fluid that can then flow throughout the void as the item is shaken or moved. This could simulate a “snow-globe” effect.

Also, a mixture of embedded solid objects and decorative voids could be used. A plurality of spherical voids could be embedded in an upper region of the composition and then colored to appear as balloons. Thereafter, a wire and a solid clown figure could be embedded in a lower portion of the same composition such that it appears that the clown is holding the balloons.

If a more solid or rigid composition is used, a light emitting diode (LED) or other light source could be embedded in the decorative void and connected to a local battery or other power source. The air freshener, candle, or other item could then be used as a night light. An almost endless variety of such configurations could be implemented within the scope of the present invention.

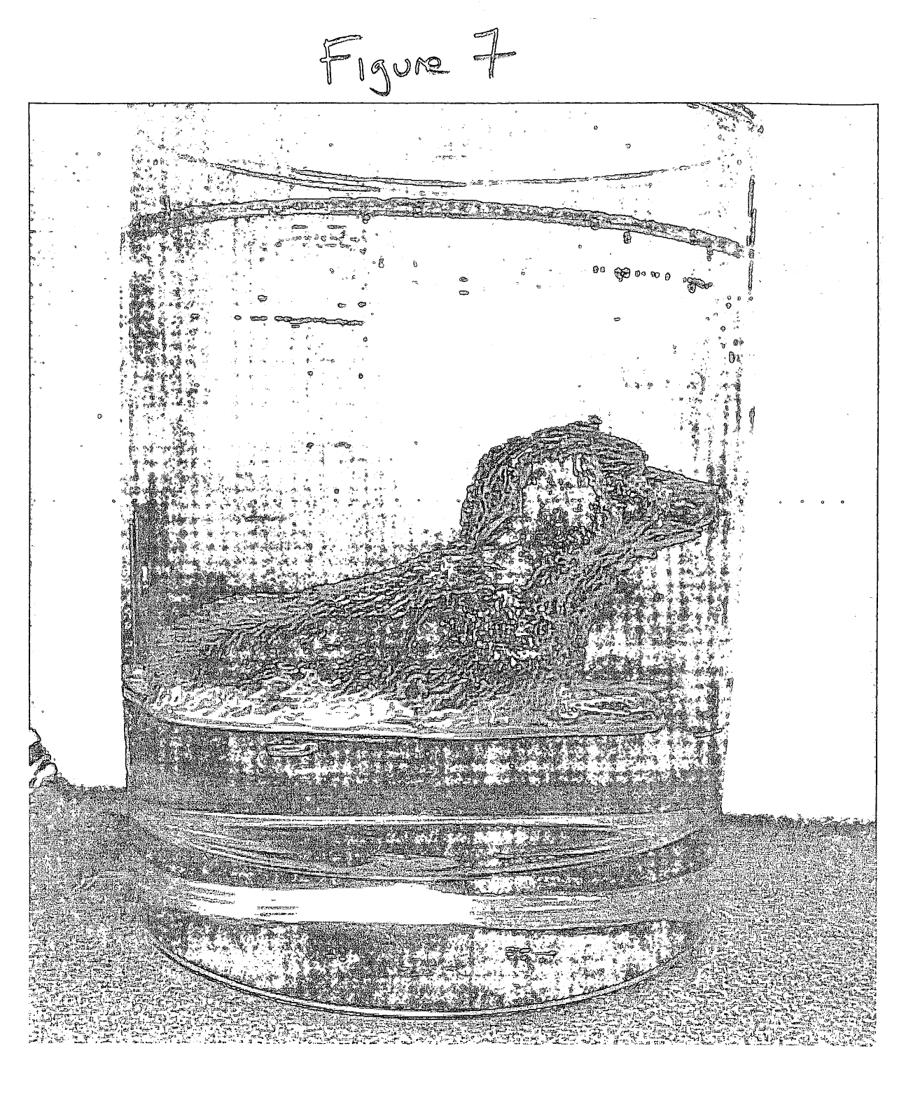

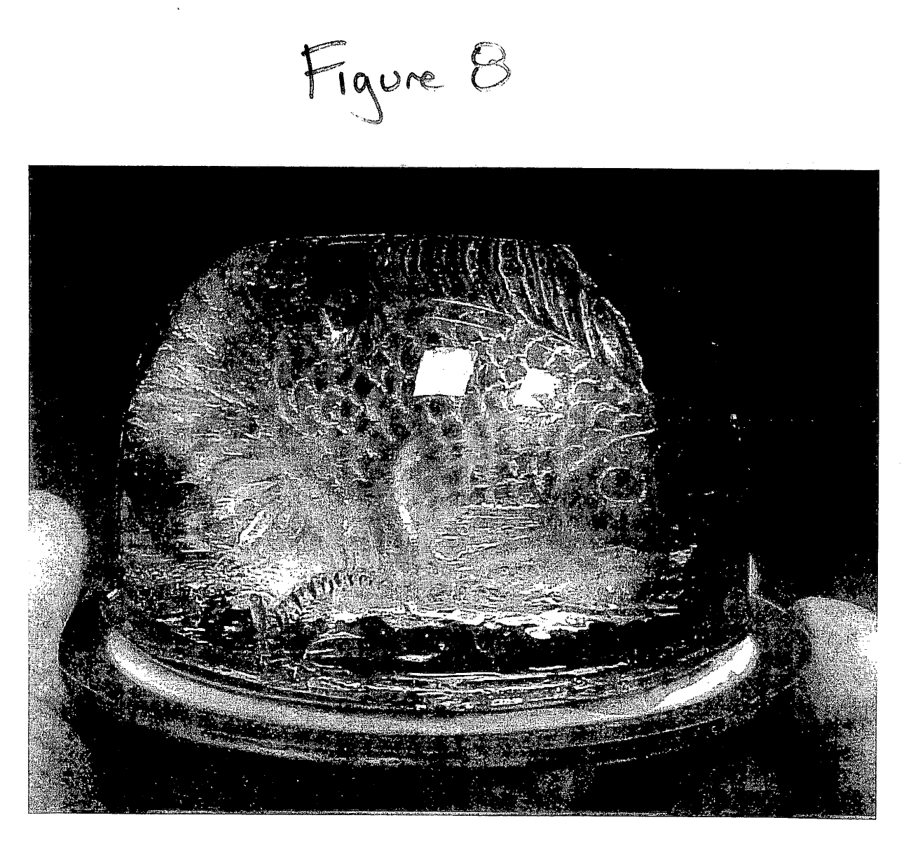

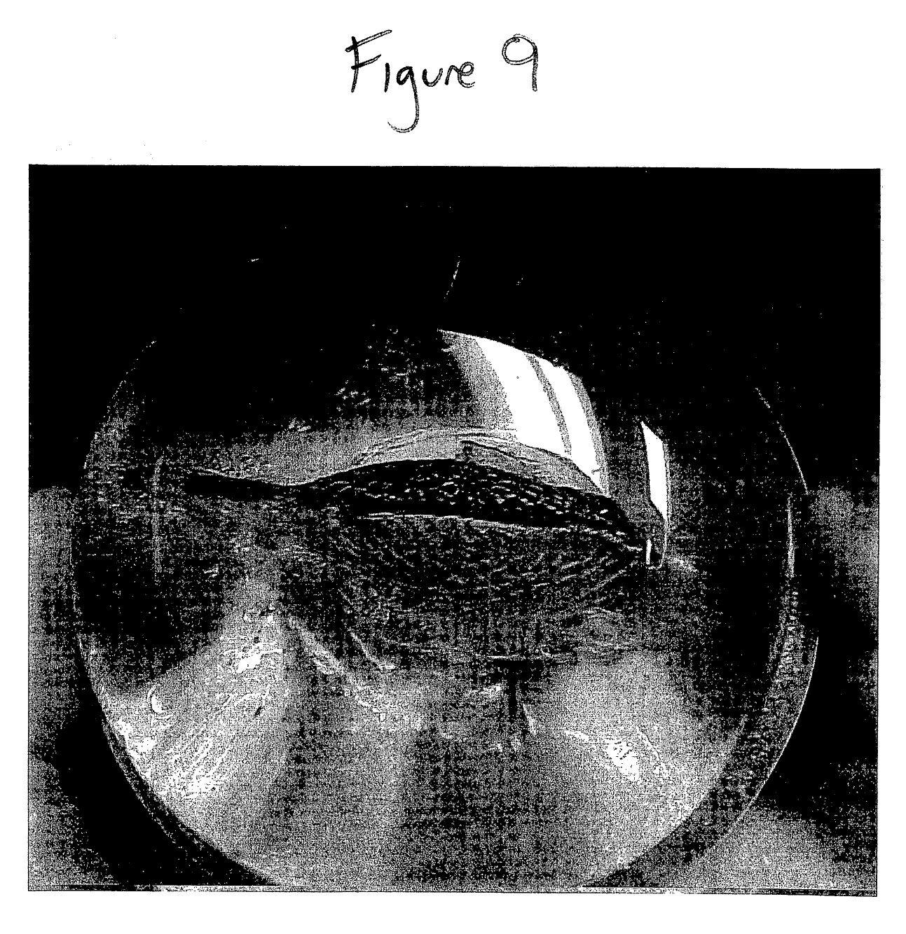

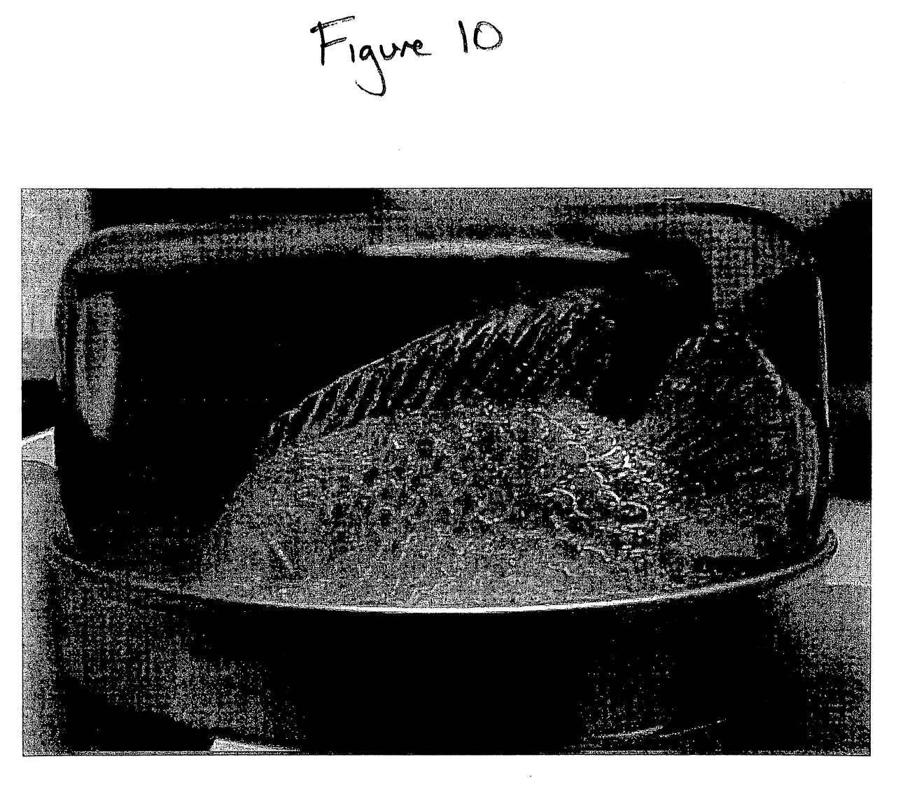

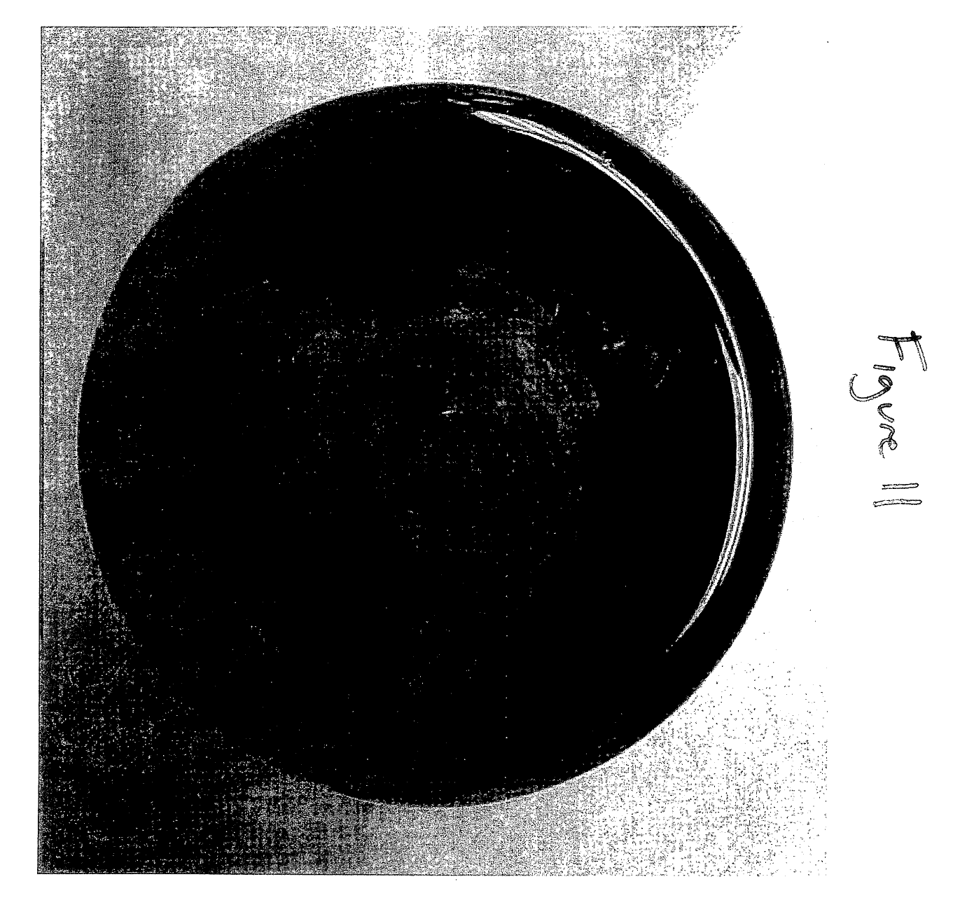

As mentioned above, a decorative object of any shape could be used with the present invention to create a decorative void in a gel-based or solid item. FIG. 6 shows the front view of an item with a void in the shape of a cat. FIG. 7 depicts a container with an item including a decorative void in the shape of a dog. FIGS. 8-10 depict various views of an item with a decorative void in the shape of a fish. Finally, FIG. 11 depicts a rose-shaped decorative void which may exist in a tinted, rose-colored composition.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

1-20. (canceled)

21. A decorative gel, comprising:
a substantially clear, elastic composition, wherein said substantially clear, elastic composition has been molded, thereby defining:
an external surface, wherein said external surface is defined by a pouring mold; and
an internal surface, wherein said internal surface is defined by a decorative object and defines a void within said decorative gel, further wherein said decorative object has imparted surface detail to the internal surface, further wherein said external surface is shaped differently than said internal surface.

22. The decorative gel of claim 21, wherein said internal surface is visible through said external surface.

23. The decorative gel of claim 21, wherein said decorative gel is an elastic water-based gel.

24. The decorative gel of claim 21, wherein said internal surface defines a first void diameter and a second void diameter, further wherein said first void diameter is wider than said second void diameter.

25. The decorative gel of claim 24, wherein said second void diameter is more proximal to said external surface than said first void diameter.

26. The decorative gel of claim 24, wherein said first void diameter is more proximal to said external surface than said second void diameter.

27. The decorative gel of claim 21, wherein said substantially clear, elastic composition is water-based.

28. The decorative gel of claim 27, wherein said substantially clear, elastic composition comprises:
from about 50% to about 99% water;
from about 0.1% to about 25% water-compatible gellant;
from about 0.1% to about 25% fragrance.

29. The decorative gel of claim 21, wherein said substantially clear, elastic composition is oil-based.

30. The decorative gel of claim 29, wherein said substantially clear, elastic composition comprises:
from about 50% to about 99% oil;
from about 0.1% to about 45% oil-compatible gellant;
from about 0.1% to about 85% fragrance.

31. The decorative gel of claim 21, wherein said void is filled with a composition that is different from said substantially clear, elastic composition.

32. The decorative gel of claim 21, wherein said substantially clear, elastic composition is essentially colorless.

33. The decorative gel of claim 21, wherein said internal surface is coated with glitter or coloring.

34. The decorative gel of claim 21, wherein said decorative gel is supported or contained within a container.

35. A method of making an item with a decorative void, comprising:
providing a quantity of substantially clear, elastic composition in a mold;
inserting at least one decorative object into said substantially clear, elastic composition;
allowing said substantially clear, elastic composition to at least partially cure; and
removing said decorative object.

36. The method of claim 35, wherein said removing is accomplished by cutting said substantially clear, elastic composition.

37. The method of claim 36, further comprising
adding additional substantially clear, elastic composition to an area cut by said cutting step.

38. The method of claim 35, wherein said object has undercut features.
